(19) United States
(12) Patent Application Publication
(10) Pub. No.: US 2006/0275333 A1
(43) Pub. Date: Dec. 7, 2006

(54) Composition with beads for treatment of contact dermatitis

(75) Inventors: John Olin Trimble, Texarcana, TX (US); Christopher Michael Brisco, Wake Village, TX (US)

Correspondence Address: JONES DAY, 77 WEST WACKER, CHICAGO, IL, 60601-1692, US

(73) Assignee: Humco Holding Group, Inc., Texarcana, TX (US)

(21) Appl. No.: 11/446,678

(22) Filed: Jun. 5, 2006

Related U.S. Application Data
(60) Provisional application No. 60/687,583, filed on Jun. 3, 2005.
(60) Provisional application No. 60/699,242, filed on Jul. 14, 2005.

(51) Int. Cl. A61K 8/49; A61K 31/537

(52) U.S. Cl. 424/401; 514/231.2; 424/70.31

(57) ABSTRACT

A treatment for urushiol induced pain and itching is provided for in a topical wash. A method is provided for applying, foaming, and removing a composition of substances to the effected area. The composition comprises a pramoxine compound in combination with a water-soluble nonylphenol ethoxylate and an inert foaming agent, preferably ammonium lauryl sulfate. It is believed that this combination stabilizes the neuronal membrane of the nerve endings with which it comes into contact, binds to the available urushiol rendering it inactive, and removes the inactive urushiol. The inert foaming agent is included to assist in the removal of the urushiol. The composition also can include one or more of a foam stabilizer, a preservative, an emulsifier, a moisturizer, and a pH adjuster. In one embodiment, the composition can comprise a quantity of cleansing beads to facilitate cleansing of the dermatitis-affected area.

This application claims the benefits of the provisional application filed by the same inventors on Jun. 3, 2005, Ser. No. 60/687,583, and the provisional application filed by the same inventors on Jul. 14, 2005, Ser. No. 60/699,242, under 35 U.S.C. §119(e)

### I. FIELD OF THE INVENTION

The present invention relates to treatments for contact dermatitis associated with poison ivy/poison oak.

### II. BACKGROUND OF THE INVENTION AND PRIOR ART

Poison oak and its eastern counterpart poison ivy are the bane of millions of campers and others who enjoy the great outdoors. They are the most common cause of allergic reactions in the United States. Each year 10 to 50 million Americans develop an allergic rash after contact with these poison plants. There are probably more myths about these plants than any other native species. Poison oak and poison ivy do not spare age or sex. Each year thousands of people are afflicted with moderate to severe dermatitis from touching the foliage of these plants. Poison oak and poison ivy account for an estimated ten percent of lost time in the U. S. Forest Service. Hundreds of fire fighters who battle summer and fall blazes in California's coastal ranges are so severely affected that they are unable to work. People who breathe in the smoke and soot may develop serious inflammation of respiratory mucous membranes. Poison oak injuries are covered by Worker's Compensation Insurance in California. It has been reported that the monetary cost of the affliction is approximately one percent of the state's workers' compensation budget.

The oleoresin urushiol causes an allergic reaction. Urushiol is a sticky oil containing catechols and other phenolic resins that does not stimulate antibody formation but reacts selectively in vitro with an antibody. It triggers an allergic reaction when it comes into contact with the skin. A person can be exposed to urushiol directly or by touching objects that have come into contact with the sap of one of the poison plants. This sap has five of these catechols. The sap exudes only when the plant is damaged. The person who touches an intact plant will not undergo any allergic reaction. The fragile stems and leaves are easily damaged by wind and animals so that a seemingly intact plant that is touched carefully may cause sensitization as a result of residual urushiol. The roots also exude the sap when torn from the ground.

According to Guin J D and Beaman J H, “Toxicodendrons of the United States,” Clin Dermatol 1986; 4:137-148, poison ivy and poison oak are now classified in the genus Toxicodendron which is readily distinguished from Rhus. There are two species of poison oak. There are also two species of poison ivy. There are nine subspecies of T. radicans. One species of poison sumac occurs in the United States. The principle function of secondary chemicals in this genus is presumably as a defense against herbivores. People worldwide are familiar with the compounds. Oleoresins cause cell-mediated contact dermatitis. The genus including poison-ivy is the most studied of the genera. These compounds are of chemical interest and they hold great promise in the search for new medicinal and commercial agents. They are the main causes of allergic skin rashes in North America. Poison oak and poison ivy are most common with oak being prevalent in western North America and ivy being prevalent in eastern North America. The name Toxicodendron is fitting as it describes two distinctive attributes of these plants. Toxic refers to the fact that significant contact with these plants often causes severe symptoms and dendron refers to the tentacle-like nature of the branches that seem designed to promote contact with all who come too close. All parts of these plants contain the toxic resinous oil urushiol that is responsible for the plants' allergenic properties.

U.S. Pat. No. 6,423,746 discloses a method of treating urushiol-induced dermatitis by applying a composition of a nonyl phenyl ethoxylate and sodium lauryl sarcosinate. The patent reports that this basic composition was known for many years as a hand scrub product but was never recognized to be effective in a treatment for urushiol-induced dermatitis. The composition as recited in the patent claims also may contain a second nonyl phenyl ethoxylate, acetylated lanolin alcohol, acetylated polyethylene granules, water, EDTA, a foam stabilizer, and a thinning agent.

Zanfel® Poison Ivy Wash is a commercially available product that claims to effectively remove the symptoms associated with poison ivy by removing urushiol. This commercial composition comprises at least one ethoxylate which, in combination with sodium lauryl sarcosinate, is stated to surround and remove the toxin from the dermal layers so that the body can begin healing. It is believed that this product is covered by the aforementioned U.S. Pat. No. 6,423,746.

### II. OBJECTS OF THE INVENTION

It is an object of the present invention to provide a method for treating dermatitis associated with contact with urushiol.

It is an object of the present invention to provide a composition for treating dermatitis associated with contact with urushiol.

It is a yet further object of the present invention to provide a composition for treatment of contact dermatitis associated with urushiol contact which composition facilitates removal of the urushiol from the affected area.

### IV. SUMMARY OF THE INVENTION

The above objects of the invention are provided for in a topical wash suitable for treatment of urushiol induced contact dermatitis. A method is provided for applying, foaming, and removing a composition of substances to the effected area. The composition comprises a pramoxine compound in combination with a nonylphenol ethoxylate and an inert foaming agent in a pharmaceutically acceptable vehicle for topical application. It is believed that this combination stabilizes the neuronal membrane of the nerve endings with which it comes into contact, binds to the available urushiol to block its allergenic properties, thereby rendering it inactive, and removes the inactive urushiol. The affinity of the urushiol for nonylphenol ethoxylate appears to prevent the urushiol induced increase in skin irritation and inflammation when measured at 4, 12, 24, and 48 hour time points. An inert foaming agent, preferably ammonium lauryl sulfate, is included to facilitate removal of the urushiol. In one embodiment, the composition is formulated with a portion of beads of the type used in dermatological cleansing compositions. The composition may also include any one or more of a foam stabilizer, chelating agent, preservative, emulsifier, moisturizer, and pH adjuster. Water maybe used as the carrier.

### V. DETAILED DESCRIPTION OF THE INVENTION

The chemical structure of a urushiol compound is shown below:

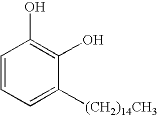

Urushiol is a general term applied to the toxic substance in the sap causing allergic contact dermatitis in people. It is actually a mixture of potent benzene ring compounds with a long side-chain of 15 or 17 carbon atoms. The side chain may be saturated or unsaturated with double bonds, as reported in Dawson, C. R., “The Toxic Principle of Poison Ivy and Related Plants,” Recent Chemical Progress, 15:39-53 (1954), and Dawson, C. R., “The Chemistry of Poison Ivy,” Transactions of the New York Academy of Sciences, 18:427-443 (1956). The remarkable immune reaction and specificity of the catechol molecule is determined by the long side-chain, as reported in Baer, H., Watkins, R. C., Kurtz, A. P., Byck, J. S., and Dawson C. R., “Delayed Contact Sensitivity to Catechols,” Journal of Immunology, 99:370-375 (1967). Poison oak urushiol contains mostly catechols with 17 carbon side-chains and poison ivy and poison sumac contain mostly 15 carbon side-chains.

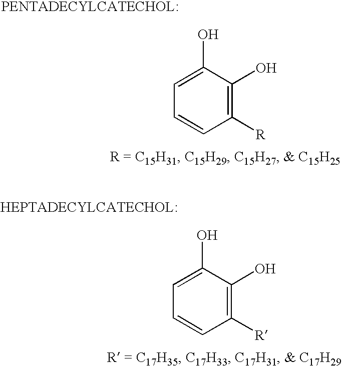

Approximately 80-90 percent of adult Americans will get a rash if they are exposed to 50 micrograms of purified urushiol, as reported in Epstein, W. L., Baer, H., Dawson C. R., and Khurana, R. G., “Poison Oak Hyposensitization: Evaluation of Purified Urushiol,” Archives of Dermatology, 109:356-360 (1974). This is indeed a minute amount when one considers that one grain of table salt weighs about 60 micrograms. Urushiol residue on the skin is difficult to wash off and may be spread by scratching. It is not spread through blister fluids. It is a relatively stable compound and can retain its potency for years in the absence of oxidation. Herbarium specimens 100 years old have been known to cause dermatitis. It is readily transferred from contaminated clothing and objects. To make matters worse it readily penetrates the epidermal layer of the skin where it binds to proteins of deeper skin cell membranes. Before the protein bond can occur the catechol is oxidized to a more reactive quinone in which the two OH groups are replaced by double-bonded oxygens.

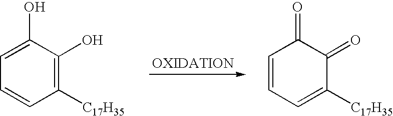

The inventors herein have found that a composition comprising a pramoxine compound, a nonylphenol ethoxylate, and an inert foaming agent in a pharmaceutically acceptable vehicle for topical application provides effective treatment for urushiol induced contact dermatitis.

Pramoxine is the common name of 4-[3-(4-Butyoxyphenoxy) propyl] morpholine. Pramoxine typically is used in the form of pramoxine HCl. Pramoxine HCl has been well-documented to provide beneficial topical anesthetic effects with a low incidence of sensitivity or reactions. (Yosipovitch, Gil MD and Maibach, Howard I. MD, Journal of the American Academy of Dermatology, August 1997 (37: 278-80), “Effect of topical pramoxine on experimentally induced pruritus in humans”) Pramoxine HCl has been shown to perform significantly better than placebo: 93% of patients treated with pramoxine HCl experienced itch reduction versus 36% for those treated by placebo; itch duration was reduced by 71% among those given pramoxine; pramoxine HCl has an onset of action of 2-6 minutes; and pramoxine HCl lasts several hours for most patients (“Tronothane (Pramoxine)”, The Medical Letter, Vol. 23, No. 23, Nov. 13, 1981, p. 100).

It has been found that certain water-soluble nonylphenol ethoxylate compounds appear to provide a protective effect. Nonylphenol ethoxylate is a common name for the compound 2-[2-(4-nonylphenoxy)ethoxy]ethanol. This compound is available from Rhodia under the name Igepal CO-630 and from BASF under the name Iconol NP-9.

The efficacy of nonylphenol ethoxylate on urushiol-induced dermatitis was evaluated by ELISA and MTT techniques. The MTT evaluation was based on a technique first described in Mosmann, T., “Rapid calorimetric assay for cellular growth and survival: application to proliferation and cytotoxicity assays,” J. Immunol. Methods, Dec. 16, 1983, 65(1-2):55-63. The analysis is based on the compound [3-(4,5-dimethylthiazol-2-yl)-2,5-diphenyltetrazolium bromide], commonly referred to as MTT. As reported by Mosmann, mitochondrial dehydrogenase enzyme from viable cells can cleave the tetrazolium rings of the pale yellow MTT and form dark blue formazin crystals. The crystals cannot permeate cell membranes, and thus tend to accumulate within healthy cells. Solubilization of the cells by the addition of a detergent results in the liberation of the crystals, which then are also solubilized. The formazin is extracted with isopropanol and measured spectrophotometrically. The intensity of the purple color is directly proportional to the metabolic activity of the cells and inversely proportional to the toxicity of the test material.

When urushiol is applied to human skin it can elicit the synthesis and release of several key inflammatory cytokines, including IL-1α, IL-1β, IL-6, and IL-8. Release of these cytokines also has been noted in cultured keratinocyte models after the application of urushiol. IL-1α and IL-6 are synthesized and stored in keratinocytes and have been identified as mediators of skin irritation and inflammation. Release of these two cytokines can be measured directly in tissue culture media by a calorimetric enzyme linked immunosorbent assay (ELISA). In such measurements, antibodies covalently linked to a solid support will bind any IL-1α or IL-6 present in spent culture media samples. A second antibody that is covalently attached to an acetylcholinesterase enzyme will in turn detect the bound cytokines. Upon addition of an appropriate color substrate the acetylcholinesterase enzyme will in turn detect the bound cytokines. Upon addition of an appropriate color substrate the acetylcholinesterase enzyme will generate a colored end product that can be measured spectrophotometrically.

Testing of the invention herein was conducted using an artificial skin product designed for toxicity testing and sold under the name Epiderm® by Mat Tek Corporation of Ashland, Massachusetts. This artificial skin product consists of normal human-derived epidermal keratinocytes that have been cultured to form a multilayered, highly differentiated model of the human epidermis. Ultrastructural analysis has revealed the presence of keratohyalin granules, tonofilament bundles, desmosomes, and a multi-layered stratum corneum containing intercellular lamellar lipid layers arranged in patterns characteristic of in vivo epidermis. Markers of mature epidermis specific differentiation such as profilaggrin, the K1/K10 cytokeratin pair, involucrin, and type I epidermal transglutaminase have been localized in this product. The product is also mitotically and metabolically active.

The Epidermic tissue samples were stored at 2-8° C. until used. The analyses were conducted with an ELISA test kit from Cayman Chemicals of Ann Arbor, Michigan, with all buffers and reagents being prepared according to the kit's instructions and allowed to come to room temperature. Prior to use, the tissues were removed from the agarose shipping tray and placed into a 6-well plate containing 1.0 ml of assay medium from the test kit, at 37±2° C. The tissues were allowed to incubate for one hour at 37±2° C. and 5±1% CO₂. After this initial incubation, the assay medium was replaced with 1 ml of fresh medium at 37±2° C. Each test sample was then treated with 100 μl of test material by applying the test material directly to the tissue surface. The tissues were incubated at 37±2° C. and 5±1% CO₂ in cell culture medium from the test kit. The test materials were a urushiol control lotion comprising 0.05% 2-deoxy-urushiol, and a test composition comprising the control lotion with the further addition of 0.2% w/v nonyl phenyl ethoxylate. At intervals of 4, 12, 24, and 48 hours, the cell culture media were collected and replaced with 1 ml of fresh media. The collected culture media were stored at −75±5° C. until analyzed for IL-1α and IL-6 levels. The IL-1α and IL-6 levels were determined by comparison with a standard curve generated from samples having known concentrations of these two cytokines. A regression analysis was performed to establish the line that best fit those points. Absorbance values for the test materials and untreated samples were then used to estimate the amount of cytokine present in each sample.

The results demonstrated that the test composition with nonylphenol ethoxylate appears to prevent the urushiol-induced increase in several key inflammatory cytokines, specifically IL-1α at the 4 and 12 hour time points, and IL-6 at the 24 and 48 hour time points, as shown in Tables I and II below

TABLE II
IL-6 Accumulation in Cell Culture Media (pg/ml)

| Treatment | 4 Hours | 12 Hours | 24 Hours | 48 Hours |
|---|---|---|---|---|
| Untreated | 2.1 ± 1.0 | 4.3 ± 1.6 | 6.6 ± 1.3 | 8.8 ± 1.6 |
| Urushiol | 1.9 ± 0.4 | 5.1 ± 0.4 | 9.4 ± 0.3* | 16.4 ± 1.4* |
| Nonylphenol | 2.2 ± 0.9 | 4.5 ± 1.4 | 7.8 ± 1.7 | 10.7 ± 2.6 |
| Ethoxylate | | | | |

TABLE I
IL-1α Accumulation in Cell Culture Media (pg/ml)

| Treatment | 4 Hours | 12 Hours |
|---|---|---|
| Untreated | 8.7 ± 2.7 | 22.7 ± 2.4 |
| Urushiol | 16.1 ± 5.2 | 32.6 ± 6.3* |
| Nonylphenol Ethoxylate | 11.1 ± 1.4 | 25.9 ± 3.0 |

Epidermal irritation often is associated with a loss of viable ketinocytes. Therefore, changes in tissue viability after exposure to urushiol and the test were assessed using the MTT assay described above. After the 48-hour time period, the tissue samples were washed with phosphate buffered saline to remove any residual test material, then transferred to a 24-well plate containing 300 μl of the assay medium from the test kit supplemented with MTT at a concentration of 1 mg/ml. The samples were allowed to incubate for 3±0.25 hours at 37±2° C. and 5±1% CO₂ Following incubation, the tissue samples were rinsed with phosphate buffered saline, blotted dry, and placed in a 24-well plate containing 2 ml of isopropanol per well. The 24-well plate was covered and allowed to incubate at room temperature for at least two hours on a rocking platform to extract the reduced MTT from the tissues. After the extraction, a 200 μl sample from each well of the isopropanol/MTT mixture was transferred to a 96-well plate and the absorbance of the sample was read at 540 nm with a plate reader using 200 μl of isopropanol as the blank.

The mean MTT absorbance value for the untreated tissues was calculated and used to represent 100% tissue viability. The individual MTT absorbance values from the tissues undergoing the various treatments was then divided by the mean value for the untreated tissues and expressed as a percent to determine the change in tissue viability caused by each treatment. It was found that treatment with nonylphenol ethoxylate also appears to prevent the urushiol induced decrease in tissue viability, as shown in Table III.

TABLE III
MTT Assay

| Treatment | Percent Viability |
|---|---|
| Untreated | 100 ± 5 |
| Urushiol | 88 ± 5 |
| Nonylphenol Ethoxylate | 99 ± 2 |

Water-soluble nonylphenol ethoxylate compounds found suitable for use in the present invention include Iconol NP-9 available from BASF and Igepal CO-630 available from Rhodia.

The composition of the present invention further comprises an inert foaming agent that preferably provides thick, dense, creamy foam with small bubble size, generates substantial foam volume, and has good cleanability. The foaming agent may be present in the amount of about 14-35% w/v. The following compounds known to be foaming agents for foams of various densities and stabilities are ranked from dense foam to loose foam as follows (“Stepan Anionic Surfactants in Personal Care”, Stepan Company, Apr. 11, 2000):

Ammonium Lauryl Sulfate/Sodium Lauryl Sulfate/triethanolamine Lauryl Sulfate/Sodium C14-C16 Olefin Sulfonate/Ammonium Laureth-1 Sulfate/Sodium Laureth-1 Sulfate/ammonium lauryl ether sulfate-2/sodium lauryl ether sulfate-2/ammonium lauryl ether sulfate-3/sodium lauryl ether sulfate-3. Ammonium lauryl sulfate is the preferred foaming agent of the present composition.

A percentage of 0.5-1.0% w/v pramoxine HCl has been approved for topical application by the FDA. The nonylphenol ethoxylate compound used in the inventive composition must be water-soluble, preferably present in the range of about 0.2-0.5% w/v. The foaming agent can be present in the range of about 14-35% w/v. The inventors have determined that a ratio of pramoxine HCl-to-nonylphenol ethoxylate-to-ammonium lauryl sulfate of about 0.5:0.1:7.0 is preferred. The amount by volume of ammonium lauryl sulfate or other inert foaming agent can vary according to the foaminess desired. The formula is not restricted to these ranges.

A suitable alkaline agent can be used to adjust the pH. A pH-balancing agent should be one that does not chemically react with the composition. The pH-balancing agent makes the overall composition approximate the slightly acid pH balance of human skin. Sodium hydroxide may be used for this purpose. Other ingredients that may be present in compositions of the present invention include foam boosters, viscosity agents, chelating agents, emulsifiers and suspending agents, and humectants or moisturizers. Ingredients that have been found suitable in the inventive composition include cocamide methyl isopropyl alcohol as a viscosity and foam builder, disodium EDTA as a chelating agent, distearyl phthalic acid amide as an emulsifier and suspending agent, glycerin as a humectant, and purified water as a carrier.

In accordance with the method of the present invention, the composition is applied to an effected area and worked over the area by a lathering motion. The composition is allowed to remain on the affected area for a sufficient amount of time for the composition to have an effect. The composition and bound urushiol are then washed away. The foaming agent facilitates the removal of the urushiol in the washing step. Experiments have demonstrated that 93% of people need only one treatment to be relieved of itching. The inventive composition reduces itch within 2-6 minutes and protects against urushiol induced inflammation for up to 48 hours.

In an alternative embodiment, the composition is formulated with a portion of beads of the type used in dermatological cleansing compositions. Standard polymer-based beads can be used, such as those made of polyethylene. In a preferred embodiment, however, jojoba beads are used to minimize the risk of irritation. In addition, it has been found that jojoba beads emulsify the bound urushiol better than other types of beads, such as polyethylene beads.

Jojoba beads used in the invention may range in size from about 150 microns to about 1200 microns; with the preferred bead size being from about 250 microns to about 420 microns. The range of proportions of beads is from about 0.5% to about 2.0% by weight; the preferred bead proportion being about 0.5% by weight.

The beads are added into the vortex at the final stage of mixing at or below 35 degrees C.; ammonium lauryl sulfate is a preferred foaming and suspending agent. Ammonium lauryl sulfate has the advantage of acting as a primary foaming agent, while having a viscosity sufficient to support the beads. The beads are chosen to be light enough to stay suspended in the composition. If a foaming agent is used that produces a less dense foam, then the beads should be selected to be of a size and density that will be supported by that foam.

There has been disclosed a composition suitable for use in the treatment of urushiol-induced itching, and a method of using the composition. Other variations and equivalents of the claimed composition and the components thereof, and methods of using the composition, will be readily apparent to those skilled in the art, and are intended to be encompassed by the claims appended hereto.

We claim:

1. A composition suitable for the topical treatment of dermatitis caused by the contact of urushiol with the skin, said topical composition comprising a pharmaceutically effective amount of a pramoxine compound, a pharmaceutically effective amount of one or more nonylphenol ethoxylate compounds, and an inert foaming agent.

2. The composition of claim 9 wherein the inert foaming agent is selected from the group consisting of ammonium lauryl sulfate, sodium lauryl sulfate, triethanolamine lauryl sulfate, sodium C14-C16 olefin sulfonate, ammonium laureth-1 sulfate, sodium laureth-1 sulfate, ammonium lauryl ether sulfate-2, sodium lauryl ether sulfate-2, ammonium lauryl ether sulfate-3, and sodium lauryl ether sulfate-3.

3. The composition of claim 2 wherein said inert foaming agent comprises ammonium lauryl sulfate.

4. The composition of claim 1 wherein said one or more nonylphenol ethoxylate compounds are present in the amount of about 0.2-0.5% w/v.

5. The composition of claim 1 wherein said foaming agent is present in the range of about 14-35% w/v.

6. The composition of claim 1 wherein the ratio of pramoxine compound:one or more nonylphenol ethoxylate compounds:foaming agent is about 0.5:0.1:7.0.

7. The composition of claim 1 wherein said topical composition further comprises a foam stabilizer.

8. The composition of claim 1 wherein said topical composition further comprises an emulsifier one or more of a chelating agent, a preservative, an emulsifier, a moisturizer, and a pH adjuster.

9. The composition of claim 1 wherein said topical composition further comprises a quantity of cleansing beads.

10. The composition of claim 9 wherein said beads are jojoba beads.

11. The composition of claim 10 wherein said beads have diameters in the range of about 150 to about 1200 microns.

12. The composition of claim 11 wherein said beads have diameters in the range of about 250 to about 420 microns.

13. The composition of claim 9 wherein said beads comprise about 0.5% to about 2.0% of said composition by weight.

14. The composition of claim 13 wherein said beads comprise about 0.5% of said composition by weight.

15. A method for the treatment of dermatitis caused by the contact of urushiol with the skin, the method comprising the steps of: providing a topical composition comprising a pharmaceutically effective amount of a pramoxine compound, a pharmaceutically effective amount of a nonylphenol ethoxylate, and an inert foaming agent; applying the composition to an affected area; foaming the composition on the affected area; permitting the composition to remain on the affected area a sufficient amount of time to enable the composition of matter to cause an effect; and, removing the composition from the affected area.

16. The method of claim 15 wherein said provided topical composition further comprises a quantity of cleansing beads.

17. The method of claim 16 wherein said beads have diameters in the range of about 150 to about 1200 microns.

18. The method of claim 17 wherein said beads have diameters in the range of about 250 to about 420 microns.

19. The method of claim 16 wherein said beads comprise about 0.5% to about 2.0% of said composition by weight.

20. The method of claim 19 wherein said beads comprise about 0.5% of said composition by weight.
